(19) United States
(12) Patent Application Publication
(10) Pub. No.: US 2024/0209038 A1
(43) Pub. Date: Jun. 27, 2024

(54) TARGETING PIEZO1 TO TREAT INHERITED AND AGE-RELATED MACULAR DEGENERATIONS

(71) Applicant: The Regents of The University of California, Oakland, CA (US)

(72) Inventors: Aparna Lakkaraju, San Francisco, CA (US); Li Xuan Tan, San Francisco, CA (US); Colin Germer, San Francisco, CA (US)

(21) Appl. No.: 18/575,450

(22) PCT Filed: Jun. 30, 2022
(86) PCT No.: PCT/US2022/073317

§ 371 Date: Dec. 29, 2023

Related U.S. Application Data
(60) Provisional application No. 63/217,545, filed on Jul. 1, 2021.

(51) Int. Cl. C07K 14/435; A61K 9/00; A61K 38/00; A61P 27/02; C12N 15/113

(52) U.S. Cl. CPC C07K 14/43518; A61K 9/0048; A61P 27/02; C12N 15/1138; A61K 38/00; C12N 2310/14; C12N 2310/141; C12N 2310/531

(57) ABSTRACT

Provided herein are compositions and methods for treating macular degeneration using agents that inhibit Piezo1.

### CROSS REFERENCE TO RELATED APPLICATIONS

This application is a U.S. National Stage of PCT/US2022/073317, International Filing Date Jun. 30, 2022, which claims the benefit of Provisional Application No. 63/217,545, filed Jul. 1, 2021, the disclosures of which are incorporated herein by reference for all purposes.

### REFERENCE TO A “SEQUENCE LISTING AS A TEXT FILE

The Sequence Listing written in file Sequence-Listing.txt created on Dec. 14, 2023, 2,238 bytes, machine format IBM-PC, MS-Windows operating system, is hereby incorporated by reference in its entirety for all purposes.

### BACKGROUND

Age-related macular degeneration (AMD), a complex disease that destroys central high-resolution vision, is the most common cause of permanent blindness in older adults. Global AMD prevalence is projected to exceed 200 million by the year 2040, primarily due to lack of treatments for non-neovascular or “dry” AMD, which affects ˜90% of patients (Dolgin, 2017; Wong et al., 2014). The primary site of injury in dry AMD is the retinal pigment epithelium (RPE), a monolayer of postmitotic polarized cells that performs numerous functions essential for photoreceptor health and for vision (Caceres and Rodriguez-Boulan, 2020; Lakkaraju et al., 2020). Clinical features of dry AMD include RPE abnormalities and sub-RPE and sub-retinal deposits called drusen (Handa et al., 2019; Wong et al., 2014). In late-stage dry AMD or geographic atrophy (GA), progressive RPE degeneration leads to the loss of RPE in focal patches that gradually enlarge and coalesce. RPE atrophy is accompanied by degeneration of the neighboring choriocapillaris and photoreceptors, resulting in irreversible visual dysfunction (Bonilha et al., 2020; Handa et al., 2019; Schmitz-Valckenberg et al., 2016). Although RPE atrophy is the defining feature of dry AMD, precisely how RPE injury and drusen deposition promote GA remains poorly understood. Further, very little is known about the role of mechanical stress in RPE dysfunction

Piezo1, is a Ca²⁺-permeable non-selective mechanosensing ion channel that was first identified a decade ago (Coste et al., 2010). Signaling cascades activated by Piezo1 have since been shown to regulate diverse pathophysiological processes including vascular development (Ranade et al., 2014), cell fate specification (Pathak et al., 2014) and migration (Hung et al., 2016), neuronal regeneration (Song et al., 2019) and degeneration (Velasco-Estevez et al., 2020), inflammation and the immune response (Solis et al., 2019). Piezo1 activation also leads to nuclear remodeling (Nava et al., 2020) and extrusion of live epithelial cells (Eisenhoffer et al., 2012). Further, a notable feature of Piezo1 is its ability to respond to both extrinsic and intrinsic stimuli.

The Piezo family of mechanotransducers includes Piezo1 and Piezo2, large 2,500-2,800 amino acid proteins with 26-40 predicted transmembrane helices. Piezo1 is ubiquitously expressed, whereas Piezo2 is mainly found in somatosensory neurons. Piezo1 is organized as a 900 kDa homotrimeric propeller structure and a central pore formed by the C-terminals of each monomer. The central pore determines channel conductance and ion selectivity. Piezo channels are activated by stretch, shear stress, osmotic shock, and other kinds of mechanical stimulation. These channels show very fast activation and inactivation kinetics, with recovery in the range of 100 milliseconds. Piezo1 and Piezo2 do not share sequence or structural homology with other known mechanosensors such as the Transient Receptor Potential (TRP) cation channels.

Piezo1 function can be modulated by extracellular matrix components: activation of Piezo1 by extracellular beta-amyloid plaques in Alzheimer's disease models promotes neurodegeneration (Velasco-Estevez et al., 2020) and ECM stiffening in gliomas activates Piezo1 to drive tumor growth (Chen et al., 2018). In addition, Piezo1 function is strongly coupled to membrane cholesterol and ceramide content (Ridone et al., 2020; Shi et al., 2020). Piezo1 signaling also activates genetic feedback loops that increase its expression (Ridone et al., 2019). Abnormal Piezo1 signaling also causes actomyosin contraction and cell extrusion from the epithelial monolayer by activating sphingosine-1-phosphate receptor and Rho kinase (Eisenhoffer et al., 2012).

### BRIEF SUMMARY OF ASPECTS OF THE DISCLOSURE

This section features certain aspects of the disclosure and is not provided as a comprehensive summary of all aspects of the disclosure.

In one aspect, the disclosure provides a method of treating or preventing macular degeneration in a subject, the method comprising administering a Piezo1 inhibitor to the subject. In some embodiments, the Piezo1 inhibitor is a peptide comprising amino acid sequence SEQ ID NO:1, or a variant thereof having 1, 2, 3, 4, or 5 mutations relative to the sequence of SEQ ID NO:1. In some embodiments, the peptide is administered in the form of an expression vector that encodes the peptide. In some embodiments, the Piezo1 inhibitor is an shRNA, siRNA, or miRNA that specifically targets Piezo1. In some embodiments, the shRNA, siRNa, or miRNA is encoded by an viral vector. In some embodiments, In some embodiments, the Piezo1 inhibitor is administered as an eye drop formulation. In some embodiments, the Piezo1 inhibitor is administered by local injection into the eye. In some embodiments, the subject is a human. In some embodiments, the subject has age-related macular degeneration (AMD), autosomal dominant or recessive Stargardt macular degeneration, Best vitelliform dystrophy, or Cone-Rod dystrophy.

In a further aspect, the disclosure provides an eye drop formulation comprising a peptide comprising amino acid sequence SEQ ID NO:1, or a variant thereof having 1, 2, 3, 4, or 5 mutations relative to the sequence of SEQ ID NO:1.

### BRIEF DESCRIPTION OF THE DRAWINGS

FIGS. 1A-1D. 1A, Ceramide immunostaining (red); 1B, quantitation in macular and peripheral RPE cryosections from normal and AMD donors. In the results provided in FIGS. 1A and 1B, only AMD macular RPE showed increased ceramide. 1C, Piezo1 immunostaining (green); 1D, quantitation in macular RPE of normal and AMD donors. In the results provided in FIGS. 1C and 1D, Nuclear Piezo1 was increased in AMD donor RPE (dotted circles). Mean±SEM, ** p<0.005; *** p<0.0001, unpaired t-test.

FIGS. 2A-2B. Piezo activation in primary polarized RPE. 2A, Stills from live imaging of calcium dynamics using Fluo4NW (green) in polarized primary RPE grown on collagen (Top row) or collagen embedded with glass beads (bottom row). Yoda1—Piezo1 agonist; GsM—Piezo1 antagonist. 2B, quantification of FluoNW intensity in control cells (Con), cells cultured on beads (B), treated with Yoda1 (+Y), or with GsMTx-4 (+G). Mean±SEM, * p<0.05; ** p<0.005, t-test.

FIGS. 3A-3D. Cholesterol and ceramide modulate Piezo1 activation in the RPE. 3A, Stills (at 130 sec and 300 sec) from Fluo4NW live imaging (green); 3B, quantification. In the results provided in FIGS. 3A and 3B, U18 increased cholesterol and ceramide. Mean±SEM, ** p<0.005, unpaired t-test. 3C, Representative traces of whole-cell currents in primary RPE recorded in a typical patch-clamp/HSPC experiment (40 mV holding potential, 25 mm Hg pressure step for 500 mSec). Black trace—control cells; red trace—RPE treated with b-cyclodextrin to deplete membrane cholesterol. 3D, Quantification of results in C. Mean±SEM, n=4 cells.

FIGS. 4A-4E. 4A, 3D reconstructions; 4B, quantitation of mitochondrial volumes from Tom20 staining in primary RPE cultures treated or not with Yoda1 and GsMTx-4. Warmer colors indicate healthy mitochondria and cooler colors indicate fragmented mitochondria. Mean±SEM; *** p<0.0001, unpaired t-test. 4C, H3K9me3 (red; merge with DAPI appears pink) immunostaining in primary RPE cultured on collagen or collagen with silica beads±GSMTx-4 (500 nM, 3 h). 4D, Actin labeling in live primary RPE cultures. Arrow heads: actin “knots” that indicate areas of actin polymerization prior to actomyosin contraction and cell extrusion. 4E, quantitation of actin staining intensity in primary RPE cultured on collagen or collagen with beads and treated with Yoda1 and/or GsMTx-4. Mean±SEM, *, p<0.05; *** p<0.001, unpaired t-test.

FIGS. 5A-5C. Validation of the Abca4−/− mouse model to study the role of Piezo1 in RPE dysfunction. 5A, H3K9me3 (red) immunostaining in 8-month-old wild type and Abca4−/− mouse RPE flatmounts. 5B, reconstruction of mitochondrial volumes from Tom20 staining in wild type and Abca4−/− mouse RPE flatmounts. Warmer colors indicate healthy mitochondria and cooler colors indicate fragmented mitochondria. 5C, Live imaging of mouse RPE flatmounts with FluoNW (green) and SiR-actin (red).

FIGS. 6A-6B. 6A, Representative surface reconstructions of Tom20-labeled mitochondrial networks in RPE from normal and AMD donors. 6B, Quantification of fragmented mitochondria per cell from images in A. Mean±SEM, ***, p<0.0001, t-test.

FIGS. 7A-7B. 7A, 3D reconstruction from live imaging of mitochondrial volumes in mock transfected RPE transduced with mito-RFP, treated with A2E and exposed to 10% NHS to induce complement attack. RPE were treated with Simvastatin (5 μM, 16 h), T0901317 (1 μM, 16 h) or desipramine (10 μM, 3 h) prior to imaging. Color bar: cooler colors indicate increasing mitochondrial fragmentation). 7B, Quantification of fragmented mitochondria per cell from images in A. Mean±SEM, ***, p<0.0001, t-test. C, H3K9me3 (red) immunostaining in retinal cryosections from Abca4−/− mice treated with vehicle or 10 mg/mg desipramine i.p.

### DETAILED DESCRIPTION

As used in herein, the singular forms “a”, “an” and “the” include plural referents unless the content clearly dictates otherwise. Thus, for example, reference to “a polypeptide” optionally includes two or more such molecules, and the like.

The term “Piezo-type mechanosensitive ion channel component 1” or “Piezo1” as used in the present disclosure refers to a Ca²⁺-permeable non-selective mechanosensing ion channel encoded by a PIEZO1 gene (Entrez Gene ID 9780). Human PIEZO1 protein is encoded by a PIEZO1 gene cytogenetically localized to human chromosome region 16q24.3 (coding region positions hg38 chr16:88,715,605-88,784,964 on Human December 20313 (GRCh38/hg38) Assembly (according to the University of California Santa Cruz Genome Browser)). Illustrative human PIEZO1 nucleic acid sequences, e.g., for use in design of an inhibitory RNA, re available under accession numbers NM_001142864.3 and NM_001142864.4. The term “Piezo1” can refer to any variant encoded by a PIEZO1 gene. Illustrative PIEZO1 polypeptide sequences are available under UniProt entry Q92508, e.g., Q92508-1. Piezo1 is organized as a 900 kDa homotrimeric propeller structure and a central pore formed by the C-terminals of each monomer. Piezo1 channels can be activated by stretch, shear stress, osmotic shock, and other kinds of mechanical stimulation.

The term “GsMTx4” refers to a 34 amino acid peptide that inhibits cationic mechanosensitive channels (MSCs). It has six lysine residues that have been proposed to affect membrane binding. The sequence of amino acid sequence of GsMTx4 is GCLEFWWKCNPNDDKCCRPKLKCSKLFKLCNFSF (SEQ ID NO:1). GsMTx-4 is a nontoxic component of tarantula venom that reversibly blocks Piezo1 activation with a Kd of 155 nM to 2 μM when tested in whole cell recordings as described in Bae et al., Biochemistry 50:6295-300, 2011. GsMTx-4 acts as a gating modifier and stabilizes the closed state of the Piezo1 channel; and has been given orphan drug status by the US FDA to treat Duchenne muscular dystrophy.

As used herein, the term “treat” or any grammatical variation thereof (e.g., treat, treating, treatment, etc.) refers to a therapeutic treatment of a disease in a subject, as well as prophylactic or preventative measures towards the disease. A therapeutic treatment can slow the progression of the disease, ameliorate disease symptoms, improve the subject's outcome, eliminate the disease, and/or alleviate, reduce or eliminate the symptoms of the disease. Thus, beneficial or desired clinical results include, but are not limited to, alleviation of disease symptoms, diminishment of the extent of the disease, stabilization (i.e., not worsening) of the disease, inhibiting, suppressing, or delaying or slowing disease progression, or amelioration of the disease state. Those in need of treatment include those already having the disease, condition, or disorder, as well as those at high risk of having the disease, condition, or disorder, and those in whom the disease, condition, or disorder is to be prevented.

As used herein, the term “pharmaceutically acceptable carrier” refers to an excipient or diluent in a pharmaceutical composition. The pharmaceutically acceptable carrier must be compatible with the other ingredients of the formulation and not deleterious to the recipient. In the present invention, the pharmaceutically acceptable carrier must provide adequate pharmaceutical stability to the active ingredient. The nature of the carrier differs with the mode of administration. For example, for intravenous administration, an aqueous solution carrier is generally used; for oral administration, a solid carrier is preferred.

The terms “identical” or “percent identity,” in the context of two or more polypeptide or polynucleotide sequences, refer to two or more sequences or subsequences that are the same or have a specified percentage of amino acids or nucleotides that are the same (e.g., at least 70%, at least 75%, at least 80%, 85%, 90%, 91%, 92%, 93%, 94%, 95%, 96%, 97%, 98%, 99%, or higher) identity over a specified region, when compared and aligned for maximum correspondence over a comparison window or designated region as measured by manual alignment and visual inspection or using a BLAST or BLAST 2.0 comparison algorithm, which are described in Altschul et al. (1990) J. Mol. Biol. 215: 403-410 and Altschul et al. (1977) Nucleic Acids Res. 25: 3389-3402, respectively, for nucleotide sequences with default parameters; or BLASTP with default parameters for amino acid sequences. Software for BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 11, an expect threshold of 0.05, M=2, N=−3, and a comparison of both strands. For amino acid sequences, the BLASTP program uses as defaults a word size (W) of 6, an expect threshold of 0.05, and the BLOSUM62 scoring matrix (see Henikoff & Henikoff, Proc. Natl. Acad. Sci. USA 89:10915 (1989)).

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a polypeptide “corresponds to” an amino acid in the polypeptide of SEQ ID NO:1 when the residue aligns with the amino acid in SEQ ID NO:1 when optimally aligned to SEQ ID NO:1. The polypeptide that is aligned to the reference sequence need not be the same length as the reference sequence.

A “conservative” substitution as used herein refers to a substitution of an amino acid such that charge, hydrophobicity, and/or size of the side group chain is maintained. Illustrative sets of amino acids that may be substituted for one another include (i) positively-charged amino acids Lys, Arg and His; (ii) negatively charged amino acids Glu and Asp; (iii) aromatic amino acids Phe, Tyr and Trp; (iv) nitrogen ring amino acids His and Trp; (v) large aliphatic nonpolar amino acids Val, Leu and Ile; (vi) slightly polar amino acids Met and Cys; (vii) small-side chain amino acids Ser, Thr, Asp, Asn, Gly, Ala, Glu, Gln and Pro; (viii) aliphatic amino acids Val, Leu, Ile, Met and Cys; and (ix) small hydroxyl amino acids Ser and Thr. Reference to the charge of an amino acid in this paragraph refers to the charge at physiological pH.

The terms “nucleic acid” and “polynucleotide” are used interchangeably and as used herein refer to both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, and combinations thereof. The terms also include, but are not limited to, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. Nucleic acid molecules may be modified chemically or biochemically or may contain non-natural or derivatized nucleotide bases, as will be readily appreciated by those of skill in the art. The above term is also intended to include any topological conformation, including single-stranded or double-stranded forms. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

The terms “polypeptide,” “peptide” and “protein” are used interchangeably herein, unless otherwise specified, to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

Inhibitors that Target Piezo1

The disclosure provides compositions and methods that target Piezo1, which is expressed by RPE, photoreceptors, and other cell s of the retina, for treating macular degeneration, e.g., age-related macular degeneration or Stargardt inherited macular degeneration, and other retinal degeneration diseases. A Piezo1 inhibitor for the treatment of retinal degenerative diseases, e.g., macular degeneration, in accordance with the disclosure binds to Piezo1 and inhibits activation or in some embodiments, targets a Piezo1 nucleic acid to inhibit expression of Piezo1 in the retina.

An “inhibitor” can be a small molecule, a protein inhibitor such as an antibody or a small peptide, or a nucleic acid inhibitor, such as an aptamer, a triple helix molecule, an siRNA, an shRNA, an miRNA, an antisense RNA or a ribozyme, to name a few. In some embodiments, expression and/or activity Piezo1 can be inhibited in a retinal cell by modifying the genome using CRISPR/Cas9, TALENS, zinc finger nuclease (ZNFs) or other gene editing techniques. See, for example, Li et al. “Applications of genome editing technology in the targeted therapy of human diseases: mechanisms, advances and prospects,” Signal Transduction and Targeted Therapy 5(1) (2020). Thus, examples of Piezo1 inhibitors include peptide inhibitors, e.g., peptides that bind Piezo1 such as GsMtx4; inhibitory anti-Piezo1 antibodies, aptamers, and inhibitory RNAs that inhibit or suppress expression of Piezo1 in the retina.

### GsMTx4 Polypeptide and Variants

In some embodiments, a Piezo1 inhibitor for treating macular degeneration is a GsMTx4 polypeptide. This peptide selectively inhibits gating of cationic MSCs and is active as either the L or D enantiomeric forms (Suchyna et al., Nature, 2004. 430 (6996), p. 235-240; Suchyna et al., Journal of General Physiology, 2000. 115: p. 583-598). The term “GsMTx4” is used herein to refer to GsMTx4-L, GsMTx4-D, a combination of GsMTx4-L and GsMTx4-D, or a polypeptide comprising a D and L amino acids. GsMTx4 lacks stereochemical specificity and both the L- and D-enantiomers are equally effective at blocking Piezo1 activation (Suchyna, et al. Nature 430:235-240, 2004). Accordingly, both L and D enantiomers of GsMTx4 and its variants can be used for treating a retinal degeneration disease.

GsMTx4 (SEQ ID NO:1) structure has been described (see, for example, Bowman et al., Toxicon 49:249-270, 2007.) The peptide contains an Inhibitory Cysteine Knot (Ick) motif with six cysteines and as with similar neuroactive peptides, is amphipathic. GsMtx4 also has a net charge of +5, with a high lysine content.

In some embodiments, a GsMtx4 administered to a subject comprises an amino acid sequence of SEQ ID NO:1. In alternative embodiments, a variant GxMtx4 peptide is administered. As used herein, a “variant GxMtx4 peptide” inhibits Piezo1, e.g., has at least 20% or at least 30%, at least 40%, at least 50%, at least 60%, at least 70%, at least 80%, at least 90%, or greater of the inhibitory activity compared to a GxMtx4 peptide of SEQ ID NO:1. Any assay can be used to measure inhibitory activity of a variant. In preferred embodiments, electrophysiological and calcium imaging assays are used to identify functionally active variants. For example, for electrophysiological assay (Lapajne et al., 2020), dissociated RPE cells from human donors are whole cell-clamped. Steps of positive pressure (2-30 mm Hg) are applied through the patch pipette using high-speed pressure clamp to evoke the mechanosensitive current in the presence or absence of inhibitor peptides. For live imaging of calcium influx, mouse RPE flatmounts or primary RPE cultures are loaded with Fluo-4 NW (Invitrogen, F36206) for 30, treated or not with inhibitory peptides, and imaged using Nikon spinning disk confocal microscope with 60× oil immersion objective (1.49 NA). In both assays, GsMTx-4 can be used as a positive control and inhibitory activity of a variant is compared to that of GsMTx-4 SEQ ID NO:1.

In some embodiments, a variant may be administered, e.g., a polypeptide comprising GCLEFWWKCNPNDDKCCRPKLKCSKLFKLCNXaaXaaXaa (SEQ ID NO:2), in which the last three amino acids are substituted relative to SEQ ID NO:1. In some embodiments, the amino acid at position 32 is Y. In some embodiments, amino acid at position 33 is C. In some embodiments, the amino acid at position 34 is S. In some embodiments, a variant comprises a sequence GCLEFWWKCNPNDDKCCRPKLKCSKLFKLCNYSF (SEQ ID NO:3) or GCLEFWWKCNPNDDKCCRPKLKCSKLFKLCNFCF (SEQ ID NO:4).

In some embodiments, the peptide comprises additional amino acids, for example, the peptide may be 35, 36, 37, 38, or greater in length, but is often less than 50 amino acid in length.

In some embodiments, a GsMTx4 variant has at least 80% identity, at least 85% identity, at least 90% identity, or at least 95% identity to the amino acid sequence of SEQ ID NO:1. In some embodiments, a GsMTx4 comprises 1, 2, 3, 4, or 5 mutations, relative to SEQ ID NO:1. For example, in some embodiments, a variant may comprise 1, 2, 3, 4 or 5 substitutions, e.g., conservative substitutions. In some embodiments, a variant comprises 1, 2, 3, 4, or 5 substitutions at positions other than cysteine. In some embodiments, an aromatic hydrophobic amino acid residue is replaced with another aromatic hydrophobic residue. In some embodiments, a variant may have a substitution at a position other than lysine.

GsMTx4 can be produced by the procedures set forth in U.S. Pat. Nos. 7,125,847 and 7,259,145. Briefly, L-GsMTx4 can be prepared by purification of the Grammostola spatulata venom, e.g., by serial fractionation using standard chromatographic techniques, such as reverse phase high performance liquid chromatography.

Alternatively, GsMTx4, or a variant thereof, can be prepared by chemical synthesis using automated or manual solid phase methods (e.g., Fmoc chemistry or automated synthesis). Depending upon quantitative yields, production of the linear reduced peptide can be performed in either a single process or in two different processes followed by a condensation reaction to join the fragments. A variety of protecting groups can be incorporated into the synthesis of linear peptide so as to facilitate isolation, purification and/or yield of the desired peptide. Protection of cysteine residues in the peptide can be accomplished using protective agents such as triphenylmethyl, acetamidomethyl and/or 4-methoxybenzyl group in any combination.

GsMTx4, or a variant thereof, may also be prepared by recombinant DNA technology using well known techniques and numerous expression systems, including, but not limited to, mammalian, yeast, insect, or prokaryotic expression systems.

### Nucleic Acid Inhibitors

In some embodiments, the inhibitor that targets Piezo1 is an inhibitory RNA, such as an shRNA, siRNA, or miRNA that targets an endogenous Piezo1 nucleic acid. Inhibitor RNAs that target Piezo1 can be designed using well-defined principles using publicly and commercially available software. Illustrative companies that provide shRNA design algorithms and services include Thermo Fisher, InvivoGen, Biosettia, Hairpin Technologies, Horizon Discovery, Eurofins Genomics and others. For example, shRNA can be identified using the Thermo Fisher website rnaidesigner.thermofisher.com/rnaiexpress/help/shrna_enter_sequence_parameters. Publicly available software Includes software available through The Broad Institute. See, also Fakhr et al, Cancer Gene Therapy 23:73-82, 2016, which describes various parameters from different algorithms for designing functional small inhibitory RNAs. Design considerations are additionally described by Ros XB-D, Gu S. Methods 103: 157-166, 2016.

The specificity or knockdown level of an shRNA, miRNA, or siRNA that targets Piezo1 can be confirmed using real-time PCR (RT-PCR) analysis for mRNA level or ELISA assay for the protein level. Experimental controls may be run in parallel to assess inhibition of Piezo1 production. Some examples of experimental controls that may be used, include but are not limited to, a mock-infected or mock-transfected sample, an shRNA encoding a scrambled target or seed region, an shRNA targeting another gene entirely such as, housekeeping genes GAPDH or Actin, or a GFP positive control. In some embodiments, an shRNA or siRNA results in expression levels that are reduced by at least about 25%, 50%, 75%, 80%, 85%, 90%, 95%, 96%, 97%, 98%, 99%, or 100% compared to a control, such as a mock-infected or mock-transfected control.

In some embodiments, a sequence encoding the RNA may be introduced into retinal cells, e.g., RPE cells, using a gene therapy vector. Such vectors include, but are not limited to lentiviral vectors, adenovirus vectors, adeno-associated virus (AAV) vectors, poxvirus vectors, and herpes virus vectors and the like. In some embodiments, the gene therapy vector is an AAV vector.

In some embodiments, a peptide inhibitor, e.g., a GsMtx4 peptide, may be encoded by an expression vector, e.g., a plasmid vector or a viral vector such as an AAV vector, adenovirus vector, or lentivirus vector.

### Treatment of Macular Degeneration Diseases

Types of macular degeneration that can be treated with a Piezo1 inhibitor (e.g., GsMtx4) in accordance with the present disclosure include age-related macular degeneration (AMD) or inherited macular degenerations such as Stargardt macular degeneration (autosomal dominant and autosomal recessive forms), Best vitelliform dystrophy, and Cone-Rod dystrophy.

The subject to be treated may be any mammal, such as, but not limited to rodents, e.g., rats or mice, or non-human primates, but is preferably a human.

Administration of a Piezo1 inhibitor is not limited to a particular site or method of administration. For example, GsMtx4 may be administered directly to the eye, e.g., in an eye drop formulation. In some embodiments, the peptide may be administered by local injection, e.g., intraocular injection or local infusion into the eye.

In some embodiments, for example when a nucleic acid inhibitor is administered via a viral vector, or a vector encoding GsMtx4, or a variant thereof, is administered, systemic administration (e.g., parenteral, intravenous injection or infusion) or local injection or infusion may be employed.

### Dose

Dosage may vary with the severity of the retinal degeneration disease. It is to be further understood that for any particular subject, specific dosage regimens can be adjusted over time according to the individual need and the professional judgment of the person administering or supervising the administration of the compositions, and that dosage ranges set forth herein are exemplary only and are not intended to limit the scope or practice of the claimed composition.

The amount of inhibitor administered will be an “effective amount” or a “therapeutically effective amount,” i.e., an amount that is effective, at dosages and for periods of time necessary, to achieve a desired result. A desired result would include a reduction in Piezo1 activity in a target cell (e.g., an RPE cell) or a detectable improvement in a symptom associated with a macular degeneration disease, including without limitation, an improvement in AMD symptoms or signs, preferably a statistically significant improvement. In some embodiments, progression of macular degeneration is assessed by evaluating the accumulation of soft, large drusen; evaluating fundus autofluorescence; and assessing dark adaptation.

If the pharmaceutical composition is used prophylactically, a desired result would include a demonstrable prevention of one or more symptoms of macular degeneration including without limitation, a symptom or sign of macular degeneration, e.g., AMD, preferably a statistically significant prevention. A therapeutically effective amount of such a composition may vary according to factors such as the disease state, age, sex, and weight of the individual.

### Formulations

In some embodiments, Piezo1 inhibitor, such as GsMtx4 is administered in conjunction with one or more lipids. The lipids can be formulated, for example, to protect and/or enhance transport/uptake of the inhibitor. In some embodiments, the inhibitor is encapsulated in liposomes, nanocapsules, microparticles, microspheres, lipids particles, lipid vesicles and the like. The formation and use of liposomes is generally known to those of skill in the art. In some embodiments, a Piezo1 inhibitor may be encapsulated in liposomes and subjected to microfluidization, which decreases liposome size to <60 nm. Liposomes of this size administered topically (directly to the eye) can reach the RPE (Lajunen et al., 2014). In some embodiments, liposomes can be actively targeted to the RPE using either the transferrin receptor (Lajunen et al., 2014) or the low-density lipoprotein-related receptor protein (LRP) (Lakkaraju et al., 2002).

In one embodiment, a Piezo1 inhibitor is delivered in a gel or ointment delivered directly to the eye. For example, drugs may be loaded in bases such as CARBOPOL 934 or liquid paraffin listed in the US Pharmacopoeia.

In some embodiments, a Piezo1 inhibitor, e.g., GsMtx4 is administered using eye drops. For example, peptide may be dissolved in sterile-filtered aqueous-based solutions. In some embodiments, GsMTx-4 can be administered in combination with a cell penetrating peptide to increase delivery to the posterior retina (see, e.g., Cogan, et al., Invest Ophthalmol Vis Sci. 58:2578-90, 2018).

In some embodiments, a Piezo1 inhibitor is provided in a refillable eye implant that provides the ability to continuously deliver the inhibitor.

In some embodiments, a subject treated with a Piezo1 inhibitor, e.g., GsMtx4 or a variant thereof, is also treated with another therapy for macular degeneration, e.g., AMD, such as an anti-VEGF agent, including for example, an anti-VEGF monoclonal antibody, e.g., bevacizumab, tranibizumab or brolucizumab, or a biosimilar antibody thereof, or an anti-VEGF agent such as aflibercept. In some embodiments, a patient treated with a Piezo1 inhibitor may also may be treated with a bispecific antibody that targets anti-VEGF and Ang-2, e.g., faricimab. In some embodiments, a subject treated with a Piezo1 inhibitor such as GsMtx4 or a variant thereof may also be treated with a long-acting DARPin anti-VEGF agent such as abicipar. It is understood that a Piezo1 inhibitor, e.g., GsMtx4, or a variant thereof, can be administered before or after treatment with another macular degeneration therapeutic agent, e.g., a Piezo1 inhibitor can be administered days, weeks, or months before or after treatment with the other therapeutic agent.

A Piezo1 inhibitor may be applied at any interval to treat macular degeneration. For example, if a GsMTx4 polypeptide is administered topically, it may be applied daily or every other day, once a week, or monthly. In some embodiments, a GsMtx4 polypeptide administered by local injection may be administered monthly, or at intervals determined by monitoring retinal degeneration in a patient. In some embodiments, a GxMTx4 polypeptide, or variant thereof, may be formulated at a dosage of from 0.1 mg to 1 mg/dose.

Inhibitory nucleic acid sequences that target Piezo1 are administered by methods known in the art, e.g., encoded by a vector, such as an AAV vector. For example, illustrative doses for administration of AAV can be about 109-1012 vector genomes per local injection into the eye.

### EXAMPLES

### Example 1. Piezo1 Signaling in RPE

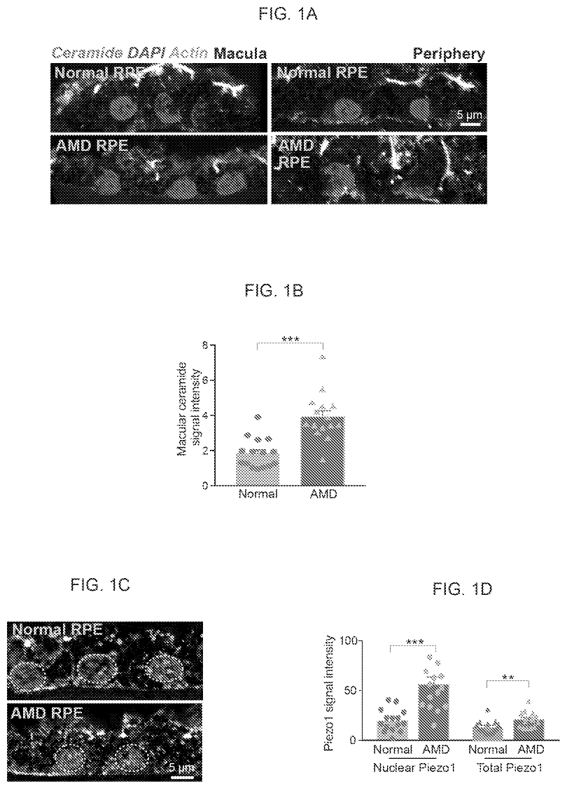

The function of Piezo1 in the retina has been largely unexplored. Recent studies have identified roles for Piezo1 in phototransduction and retinal ganglion cell degeneration (Bocchero et al., 2020; Morozumi et al., 2020). Our data demonstrated increased Piezo1 expression in AMD donor RPE and sustained Piezo1 activation in RPE with AMD-associated stressors (see, e.g., FIGS. 1C, 2, 3, 4A, and 4B).

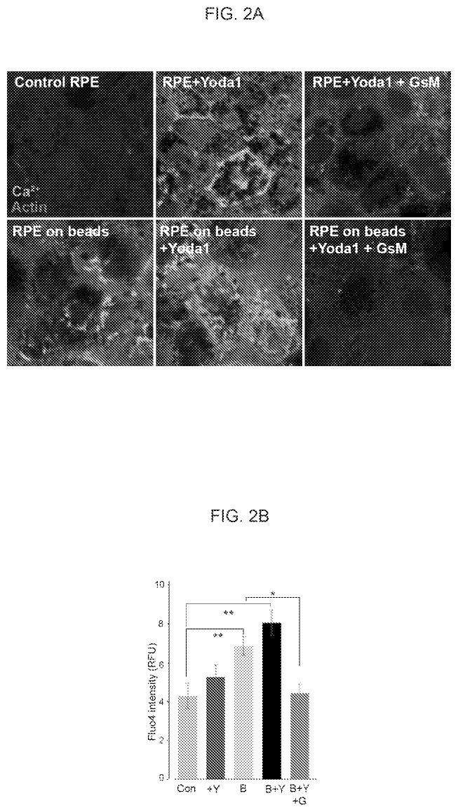

We observed that AMD donor macular RPE have more ceramide (FIGS. 1A & 1B) and nuclear Piezo1 compared to unaffected controls (FIGS. 1C & 1D). Our live imaging data showed that the Piezo1 agonist Yoda1 (Tocris, 25 μM, 1 min) induces a rapid nuclear calcium influx within seconds that propagates from the nucleus to reticular structures that resemble the ER and mitochondria, and eventually the cytosol in the RPE (FIGS. 2A & 2B). In RPE cells cultured on silica beads to simulate drusen, intracellular calcium was significantly greater than in RPE cultured on collagen. These calcium spikes were further potentiated by Yoda1 and decreased by GsMTx-4, confirming the involvement of Piezo1 in mediating calcium influx in the RPE (FIGS. 2A & 2B).

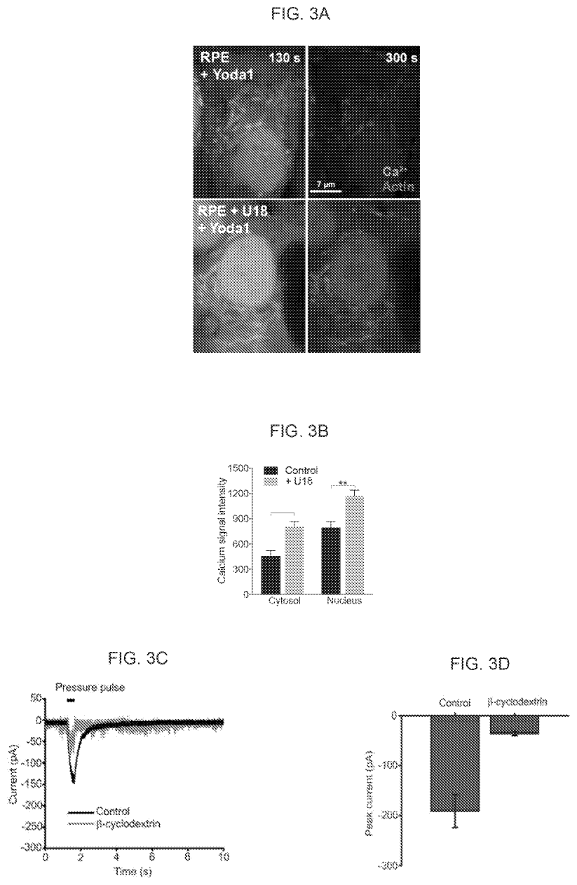

Piezo1 activity is sensitive to membrane lipid environment, and increased cholesterol and ceramide in the bilayer have recently been shown to prolong Piezo1 channel opening (Ridone et al., 2020; Shi et al., 2020). In RPE treated with the lipofuscin component A2E or the biochemical U18666A, both of which cause cholesterol and ceramide accumulation (Tan et al., 2016), we observed prolonged cytosolic and nuclear calcium even at baseline, indicative of abnormal Piezo1 activation. Moreover, after Yoda1 treatment, calcium levels remained high in all cellular compartments in RPE+U18 unlike control RPE where they returned to baseline within minutes (FIGS. 3A&3B). In agreement with cholesterol and ceramide modulating Piezo1 activity, measurement of Piezo1 currents in whole cell-clamped primary RPE showed that depleting membrane cholesterol using cyclodextrin drastically decreased peak current amplitudes (FIG. 3C&3D). These data strongly support that membrane cholesterol and ceramide increase Piezo1 activation in the RPE.

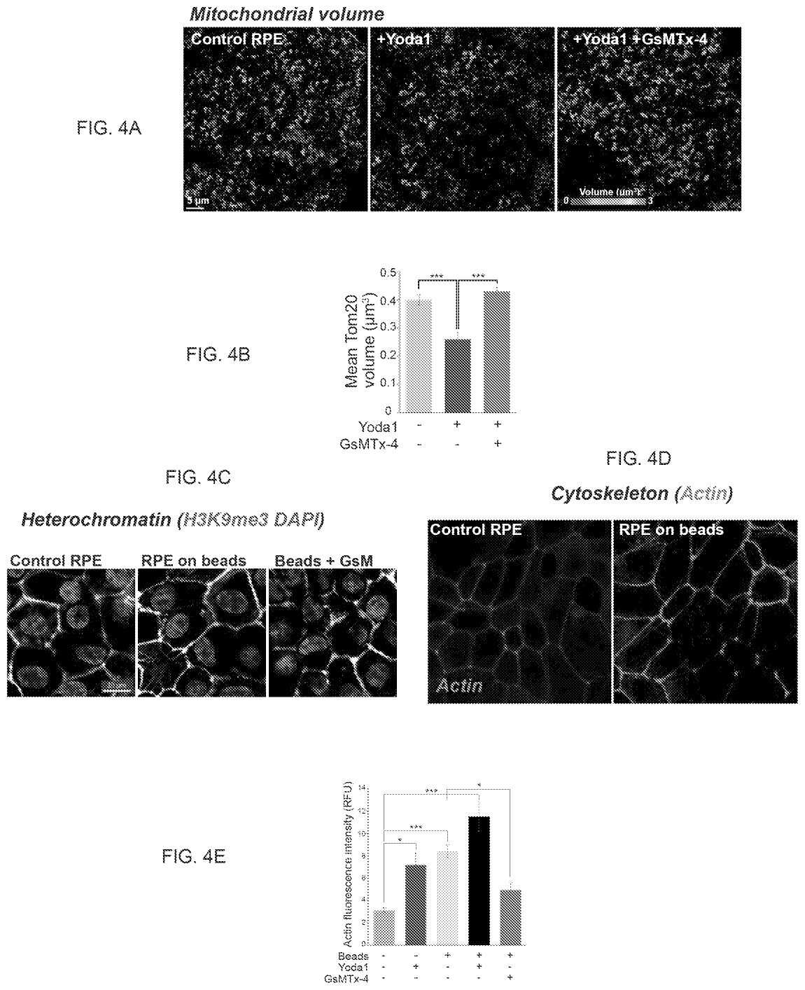

In primary polarized RPE, mechanical stress induced by culturing on beads or prolonged piezo1 activation by Yoda1 treatment resulted in loss of nuclear H3K9me3 and mitochondrial fragmentation, whereas GsMTx-4 protected RPE mitochondria (FIG. 4A-4C).

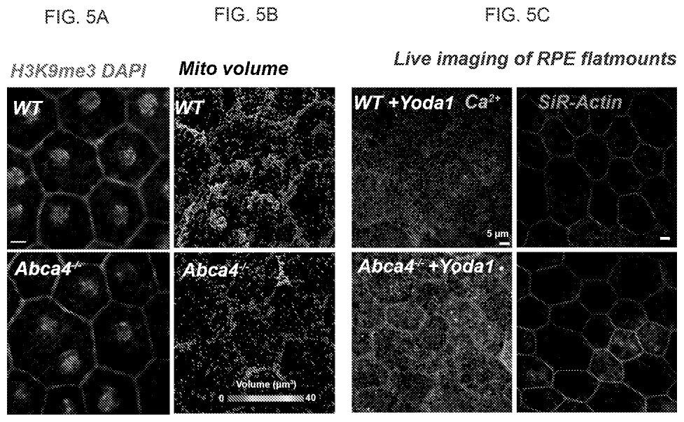

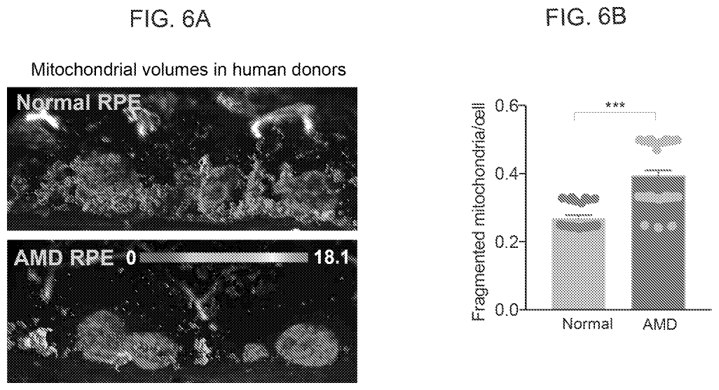

In Abca4^{-/-} mice RPE, which have increased cholesterol and ceramide (Kaur et al., 2018; Tan et al., 2016; Toops et al., 2015), we observed a striking loss of nuclear H3K9me3 (FIG. 5A), and increased mitochondrial fragmentation compared to age-matched wild type mice (FIG. 5B). Preliminary live imaging of freshly isolated RPE flatmounts showed increased calcium influx in response to Yoda1 (FIG. 5C), likely due to prolonged Piezo1 activation mediated by membrane cholesterol and ceramide. We also observed increased mitochondrial fragmentation in AMD donor RPE compared to unaffected donors (La Cunza et al., 2020) (FIGS. 6A&6B).

In AMD donors, ceramide was increased only in the macula, but not in the periphery (FIG. 1A), suggesting that ceramide (acting in concert with drusen-induced mechanical stress), could activate Piezo1 to promote RPE cell loss in the central retina, in agreement with the GA phenotype (Schmitz-Valckenberg et al., 2016). Live cell imaging of actin dynamics in polarized primary RPE cultures showed that Yoda1 caused rapid actin accumulation at cell-cell junctions, which was prevented by GsMTx-4. Actin remodeling was exacerbated in RPE grown on silica beads, likely due to mechanical activation of Piezo1 (FIGS. 4D & 4E). Live imaging of freshly isolated Abca4^{-/-} mice RPE flatmounts showed increased actin intensity after Yoda1 treatment compared to age-matched wild types (FIG. 5C).

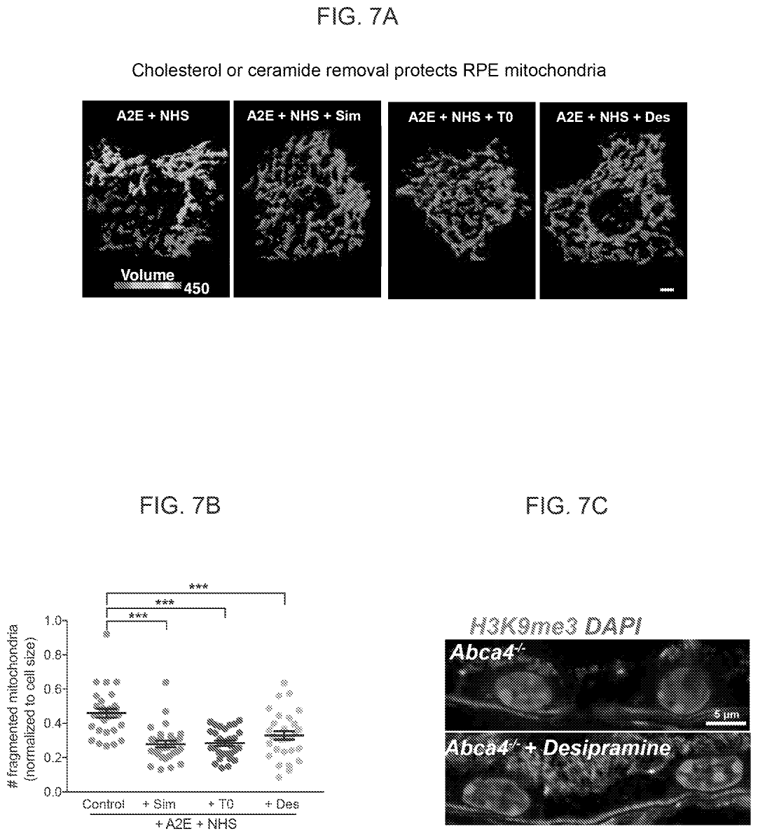

We have shown that the Liver X receptor agonist T090317 and the acid sphingomyelinase inhibitor desipramine decreased lipofuscin-induced cholesterol and ceramide accumulation, respectively, in the RPE in vitro and in vivo (Kaur et al., 2018; Tan et al., 2016; Toops et al., 2015). T0901317, desipramine, and simvastatin, which also lowers cholesterol, protect the RPE from complement-mediated mitochondrial fragmentation (FIGS. 7A & 7B) (La Cunza et al., 2020). In Abca4^{-/-} mice, desipramine administration (10 mg/kg i.p., three times/week for four weeks) restored nuclear H3K9me3 in the RPE, compared to vehicle-treated mice (FIG. 7C).

### Materials and Methods

Primary adult RPE cultures. Polarized RPE monolayers from pig eyes were generated as described (Kaur et al., 2018; Tan et al., 2016; Toops et al., 2015; Toops et al., 2014). RPE on collagen-coated transwell filters form fully polarized monolayers within two weeks, develop trans-epithelial electrical resistances (TER) of ≥400 ohm·cm² and express RPE differentiation markers such as apical Na⁺,K⁺-ATPase and intracellular RPE65 (Toops et al., 2014).

Intrinsic stressors to activate Piezo1. RPE monolayers were treated with the bisretinoid A2E to increase cholesterol and ceramide (Kaur et al., 2018; La Cunza et al., 2020; Tan et al., 2016; Toops et al., 2015), which act as intrinsic stressors that activate Piezo1.

Extrinsic stressors to activate Piezo1. To simulate drusen-mediated mechanical stress, cells were cultured on collagen-coated filters layered with 500 nm diameter monodisperse silica microspheres (Cospheric SiO2MS-2.0 0.507 μm) at a density of 40 beads/cm² in a collagen (10 μg/cm²) matrix. Our preliminary studies showed that beads are non-toxic and RPE plated on these beads form polarized monolayers within two weeks, with TERs of ˜400-500 ohm·cm²

Mice. There are no mouse models that adequately reproduce AMD phenotypes. We therefore used models that best recapitulate the phenotype of interest. We used pigmented wild-type (129S1/SvlmJ) and Abca4^{-/-} mice (Jackson Labs) because we and others have reported that Abca4^{-/-} mice RPE show an age-dependent accumulation of cholesterol and ceramide (Kaur et al., 2018; Lenis et al., 2017; Tan et al., 2016; Toops et al., 2015), which are known to prolong Piezo1 activation (Ridone et al., 2020, Shi et al., 2020). Data presented herein demonstrated that compared to age-matched wild type mice, 6-, 8- and 10-month-old Abca4^{-/-} mice RPE exhibited progressive loss of nuclear H3K9me3 and mitochondrial fragmentation, similar to that observed with Piezo1 activation in RPE monolayers.

Human donor tissue. Tissue, fundus photographs, and de-identified medical records from unaffected and AMD donors were obtained from the Lions Eye Bank of Minnesota (death to preservation time <6 hours). Exclusion criteria include diabetic retinopathy, glaucoma, and trauma to the posterior eye. Tissue will be genotyped for CFH, ARMS2/HTRA1, C2, and C3 risk variants (La Cunza et al., 2020). A minimum of 9 unaffected and 9 AMD donor retinas (central and peripheral retina/RPE/choroid/sclera were separately isolated, and cryosectioned) were analyzed (Ferrington et al., 2016; La Cunza et al., 2020).

Drugs that modulate Piezo1 activity (Xiao, 2020). Yoda1 is a small molecule gating modifier that specifically potentiates Piezo1 mechanosensitivity and slows the inactivation kinetics with EC50 of ˜10-20 μM. It interacts with the agonist transduction domains of Piezo1 and thereby increases channel opening time. Yoda1 is specific for Piezo1 and does not activate Piezo2. We treated primary RPE cultures for 6 hours with 10 μM Yoda1 (Sigma SML1558) for immunostaining/immunoblotting and biochemical experiments, or with 25 μM for 1-5 minutes for live imaging experiments. RPE cultures were treated with 500 nM GsMTx-4 (Tocris 4912100U) for 6 h.

Antibodies. All antibodies used were validated for specificity using the relevant IgG controls, cell lysates lacking the antigen, or by blocking peptides. Several of the antibodies we used are commercially available and have been used in numerous publications including our own: Piezo1 (Proteintech 15939-1-AP, 1:50); H3K9me3 (Cell Signaling 5327, 1:200); Lamin A/C (Proteintech 10298-1-AP, 1:100); TOM20 (Santacruz sc-11415, 1:200). We routinely test each new lot of commercial antibody to rule out lot-to-lot variability before using it for experiments.

High-speed live-cell imaging. The Nikon CSU-X1 dual camera platform spinning disk confocal microscopy system will be used for live imaging of primary RPE cultures and mouse RPE flatmounts. Images will be analyzed using Imaris (Bitplane) (Kaur et al., 2018; Tan et al., 2016; Toops et al., 2015). If necessary, Delta Vision OMX-SR (GE Healthcare Life Sciences) with 3D structured illumination microscopy (3D-SIM) and ring-TIRF capabilities at UCSF's Nikon Imaging Center will be used for super-resolution imaging.

Live imaging of mouse RPE flatmounts. Tissue from freshly harvested eyes will be mounted onto a Warner chamber in the presence of Fluorobrite DMEM (Gibco, A1896701) supplemented with 1% FBS (ATCC, 30-2020), 1% NEAA (Corning, 25025CI), and 4 mM L-glutamine (Gibco, 25030081) and imaged immediately using a 60× oil immersion objective (1.49 NA).

Live imaging of calcium influx. For live imaging of calcium influx, primary porcine RPE grown on Transwell filters±A2E or grown on silica beads, and mouse RPE flatmounts were loaded with Fluo-4 NW (Invitrogen, F36206) for 30 min according to manufacturer's protocol. Excised Transwell filters or mouse RPE flatmounts were mounted onto a Warner chamber in the presence of Fluorobrite DMEM (Gibco, A1896701) supplemented with 1% FBS (ATCC, 30-2020), 1% NEAA (Corning, 25025CI), and 4 mM L-glutamine (Gibco, 25030081) and imaged using Nikon spinning disk confocal microscope with 60× oil immersion objective (1.49 NA). To increase or inhibit Piezo1 channel activity, Yoda1 (25 μM) or GsMTx-4 (5 μM) was added prior to live imaging. Apart from these drugs, preincubation with BAPTA-AM (Thermofisher) was used to determine the Ca²⁺-dependence of nuclear and cytosolic signals.

Electrophysiology. Dissociated RPE cells from human donors were whole cell-clamped. Steps of positive pressure (2-30 mm Hg) were applied through the patch pipette using high-speed pressure clamp (ALA Instruments) or the cells stimulated with msec indentations of a blunt pipette tip (Physik Instrumente) to evoke the mechanosensitive current. The Piezo1-dependent current was confirmed using GsMTx-4 (Lapajne et al., 2020).

Live imaging of actin dynamics was performed using SiR-actin (1 μM, 30 min) and FLIPPER-TR plasma membrane probe in RPE with A2E or grown on silica beads±Yoda1±GsMTx-4

Other techniques. Biochemical, molecular and electrophysiological techniques have been described in recent publications (Kaur et al., 2018; La Cunza et al., 2020; Lapajne et al., 2020; Tan et al., 2016; Toops et al., 2015).

Data acquisition and analysis. Data were analyzed with either a two-tailed t-test with Welch's correction for unequal variances or one-way ANOVA with the Bonferroni multiple comparisons post-test (Prism). p<0.05 is considered significant.

Rigor and Reproducibility. All experiments were designed with appropriate controls. Each experiment will have ≥9 replicates (≥3 independent experiments with ≥3 replicates per experiment per condition).

Sex as a biological variable. Equal numbers of male and female mice were used for all experiments. We have not observed any sex-based differences in our studies to date.

### LISTING OF FULL CITATIONS FOR REFERENCES CITED HEREIN BY AUTHOR/YEAR

Adada, M., D. Canals, Y. A. Hannun, and L. M. Obeid, 2013. Sphingosine-1-phosphate receptor 2. Febs. J. 280:6354-6366.
Agarwal. P., M. Rashighi, K. I. Essien, J. M. Richmond, L. Randall, H. Pazoki-Toroudi, C. A. Hunter, and J. E. Harris. 2015. Simvastatin prevents and reverses depigmentation in a mouse model of vitiligo. J Invest Dermatol. 135:1080-1088.
Aragona, M., T. Panciera, A. Manfrin, S. Giulitti, F. Michielin, N. Elvassore, S. Dupont, and S. Piccolo. 2013. A mechanical checkpoint controls multicellular growth through YAP/TAZ regulation by actin-processing factors. Cell. 154:1047-1059.
Barrero, M. J., S. Bouc, and J. C. Izpisua Belmonte. 2010. Epigenetic mechanisms that regulate cell identity. Cell Stem Cell. 7:565-570.
Becker, J. S., D. Nicetto, and K. S. Zaret. 2016. H3K9me3-Dependent Heterochromatin: Barrier to Cell Fate Changes. Trends Genet. 32:29-41.
Benham-Pyle, B. W., B. L. Pruitt, and W. J. Nelson. 2015. Cell adhesion. Mechanical strain induces E-cadherin-dependent Yap1 and beta-catenin activation to drive cell cycle entry. Science. 348:1024-1027.
Bocchero, U., F. Falleroni, S. Mortal, Y. Li, D. Cojoc, T. Lamb, and V. Torre. 2020. Mechanosensitivity is an essential component of phototransduction in vertebrate rods. PLoS Biol. 18:e3000750.
Bonilha, V. L., B. A. Bell, J. Hu, C. Milliner, G. J. Pauer, S. A. Hagstrom, R. A. Radu, and J. G. Hollyfield. 2020. Geographic Atrophy: Confocal Scanning Laser Ophthalmoscopy, Histology, and Inflammation in the Region of Expanding Lesions. Invest Ophthalmol Vis Sci. 61:15.
Caceres, P. S., and E. Rodriguez-Boulan. 2020. Retinal pigment epithelium polarity in health and blinding diseases. Curr Opin Cell Biol. 62:37-45.
Chen, X., S. Wanggou, A. Bodalia, M. Zhu, W. Dong, J. J. Fan, W. C. Yin, H. K. Min, M. Hu, D. Draghici, W. Dou, F. Li, F. J. Coutinho, H. Whetstone, M. M. Kushida, P. B. Dirks, Y. Song, C. C. Hui, Y. Sun, L. Y. Wang, X. Li, and X. Huang. 2018. A Feedforward Mechanism Mediated by Mechanosensitive Ion Channel PIEZO1 and Tissue Mechanics Promotes Glioma Aggression. Neuron. 100:799-815 €797.
Coste, B., J. Mathur, M. Schmidt, T. J. Earley, S. Ranade, M. J. Petrus, A. E. Dubin, and A. Patapoutian, 2010. Piezo1 and Piezo2 are essential components of distinct mechanically activated cation channels. Science. 330:55-60.
Curcio, C. A., E. C. Zanzottera, T. Ach, C. Balaratnasingam, and K. B. Freund. 2017. Activated Retinal Pigment Epithelium, an Optical Coherence Tomography Biomarker for Progression in Age-Related Macular Degeneration. Invest Ophthalmol Vis Sci. 58: BIO211-BIO226.
Della Pietra, A., N. Mikhailov, and R. Giniatullin. 2020. The Emerging Role of Mechanosensitive Piezo Channels in Migraine Pain. Int J Mol Sci. 21.
Dobrosak, C., and J. H. Gooi. 2017. Increased sphingosine-1-phosphate production in response to osteocyte mechanotransduction. Bone Rep. 7:114-120.
Dolgin, E. 2017. Age-related macular degeneration foils drugmakers. Nat Biotechnol. 35:1000-1001.
Dourado, L. F. N., F. R. da Silva, C. R. Toledo, C. N. da Silva, C. P. Santana, B. L. da Costa, M. E. de Lima, and A. D. S. Cunha. 2020. Intravitreal injection of peptides PnPa11 and PnPa13, derivatives of Phoneutria nigriventer spider venom, prevents retinal damage. J Venom Anim Toxins Incl Trop Dis. 26:e20200031.
Dupont, S., L. Morsut, M. Aragona, E. Enzo, S. Giulitti, M. Cordenonsi, F. Zanconato, J. Le Digabel, M. Forcato, S. Bicciato, N. Elvassore, and S. Piccolo. 2011. Role of YAP/TAZ in mechanotransduction. Nature. 474:179-183.
Eisenhoffer, G. T., P. D. Loftus, M. Yoshigi, H. Otsuna, C. B. Chien, P. A. Morcos, and J. Rosenblatt. 2012. Crowding induces live cell extrusion to maintain homeostatic cell numbers in epithelia. Nature. 484:546-549.
Ellefsen, K. L., J. R. Holt, A. C. Chang, J. L. Nourse, J. Arulmoli, A. H. Mekhdjian, H. Abuwarda, F. Tombola, L. A. Flanagan, A. R. Dunn, I. Parker, and M. M. Pathak. 2019. Myosin-II mediated traction forces evoke localized Piezo1-dependent Ca(2+) flickers. Commun Biol. 2:298.
Etem, E. O., G. G. Ceylan, S. Ozaydin, C. Ceylan, I. Ozercan, and T. Kuloglu. 2018. The increased expression of Piezo1 and Piezo2 ion channels in human and mouse bladder carcinoma. Adv Clin Exp Med. 27:1025-1031.
Ferrington, D. A., R. J. Kapphahn, M. M. Leary, S. R. Atilano, M. R. Terluk, P. Karunadharma, G. K. Chen, R. Ratnapriya, A. Swaroop, S. R. Montezuma, and M. C. Kenney. 2016. Increased retinal mtDNA damage in the CFH variant associated with age-related macular degeneration. Exp Eye Res. 145:269-277.
Gemenetzi, M., and A. J. Lotery. 2020. Epigenetics in age-related macular degeneration: new discoveries and future perspectives. Cell Mol Life Sci. 77:807-818.
Gu, Y., T. Forostyan, R. Sabbadini, and J. Rosenblatt. 2011. Epithelial cell extrusion requires the sphingosine-1-phosphate receptor 2 pathway. J Cell Biol. 193:667-676.
Handa, J. T., C. Bowes Rickman, A. D. Dick, M. B. Gorin, J. W. Miller, C. A. Toth, M. Ueffing, M. Zarbin, and L. A. Farrer. 2019. A systems biology approach towards understanding and treating non-neovascular age-related macular degeneration. Nat Commun. 10:3347.
Haws, H. J., M. A. McNeil, and M. D. Hansen. 2016. Control of cell mechanics by RhoA and calcium fluxes during epithelial scattering. Tissue Barriers. 4:e1187326.
Hung, W. C., J. R. Yang, C. L. Yankaskas, B. S. Wong, P. H. Wu, C. Pardo-Pastor, S. A. Serra, M. J. Chiang, Z. Gu, D. Wirtz, M. A. Valverde, J. T. Yang, J. Zhang, and K. Konstantopoulos. 2016. Confinement Sensing and Signal Optimization via Piezo1/PKA and Myosin II Pathways. Cell reports. 15:1430-1441.
Ito. S., S. Okuda. M. Abc. M. Fujimoto. T. Onuki. T. Nishimura, and M. Takeichi. 2017. Induced cortical tension restores functional junctions in adhesion-defective carcinoma cells. Nat Commun. 8:1834.
Kaur. G., L. X. Tan. G. Rathnasamy, N. R. La Cunza. C. J. Germer. K. A. Toops. M. Fernandes. T. A. Blenkinsop, and A. Lakkaraju. 2018. Aberrant early endosome biogenesis mediates complement activation in the retinal pigment epithelium in models of macular degeneration. Proc Natl Acad Sci USA. 115:9014-9019.
Kennedy, D., A. Samali, and R. Jager. 2015. Methods for studying ER stress and UPR markers in human cells. Methods Mol Biol. 1292:3-18.
La Cunza, N., L. X. Tan, G. Rathnasamy, T. Thamban, C. J. Germer, K. A. Toops, and A. Lakkaraju. 2020. Apolipoprotein E isoform-specific phase transitions in the retinal pigment epithelium drive disease phenotypes in age-related macular degeneration. biorxiv doi: https citation doi.org/10.1101/2020.07.15.201723.
Lakkaraju, A., A. Umapathy, L. X. Tan, L. Daniele, N.J. Philp, K. Boesze-Battaglia, and D. S. Williams. 2020. The cell biology of the retinal pigment epithelium. Prog Retin Eye Res: 100846.
Lapajne, L., M. Lakk, O. Yarishkin, L. Gubcljak, M. Hawlina, and D. Krizaj. 2020. Polymodal Sensory Transduction in Mouse Corneal Epithelial Cells. Invest Ophthalmol Vis Sci. 61:2.
Lenis, T. L., S. Sarfare, Z. Jiang, M. B. Lloyd, D. Bok, and R. A. Radu. 2017. Complement modulation in the retinal pigment epithelium rescues photoreceptor degeneration in a mouse model of Stargardt discasc. Proc Natl Acad Sci USA. 114:3987-3992.
Li, X., L. Han, I. Nookacw, E. Mannen, M. J. Silva, M. Almeida, and J. Xiong. 2019. Stimulation of Piezo1 by mechanical signals promotes bone anabolism. Elife. 8.
Lu, Q., P. A. Scott, E. V. Vukmanic, H. J. Kaplan, D. C. Dean, and Q. Li. 2020. Yap1 is required for maintenance of adult RPE differentiation. FASEB J. 34:6757-6768.
Miyamoto, T., T. Mochizuki, H. Nakagomi, S. Kira, M. Watanabe, Y. Takayama, Y. Suzuki, S. Koizumi, M. Takeda, and M. Tominaga. 2014. Functional role for Piezo1 in stretch-evoked Ca(2)(+) influx and ATP release in urothelial cell cultures. J Biol Chem. 289:16565-16575.
Morozumi, W., S. Inagaki, Y. Iwata, S. Nakamura, H. Hara, and M. Shimazawa. 2020. Piezo channel plays a part in retinal ganglion cell damage. Exp Eye Res. 191:107900.
Mulherkar, S., and K. F. Tolias. 2020. RhoA-ROCK Signaling as a Therapeutic Target in Traumatic Brain Injury. Cells. 9.
Nava, M. M., Y. A. Miroshnikova, L. C. Biggs, D. B. Whitefield, F. Metge, J. Boucas, H. Vihinen, E. Jokitalo, X. Li, J. M. Garcia Arcos, B. Hoffmann, R. Merkel, C. M. Niessen, K. N. Dahl, and S. A. Wickstrom. 2020. Heterochromatin-Driven Nuclear Softening Protects the Genome against Mechanical Stress-Induced Damage. Cell. 181:800-817 e822.
Ohsawa, S., J. Vaughen, and T. Igaki. 2018. Cell Extrusion: A Stress-Responsive Force for Good or Evil in Epithelial Homeostasis. Dev Cell. 44:532.
Parpaite, T., and B. Coste. 2017. Piezo channels. Curr Biol. 27:R250-R252.
Pathak, M. M., J. L, Nourse, T. Tran, J. Hwe, J. Arulmoli, D. T. Le, E. Bernardis, L. A. Flanagan, and F. Tombola. 2014. Stretch-activated ion channel Piezo1 directs lineage choice in human neural stem cells. Proc Natl Acad Sci USA. 111:16148-16153.
Prosen, L., B. Markele, T. Dolinsek, B. Music, M. Cemazar, and G. Sersa. 2014. Mcam Silencing With RNA Interference Using Magnetofection has Antitumor Effect in Murine Melanoma. Mol Ther Nucleic Acids. 3:e205.
Ranade, S. S., Z. Qiu, S. H. Woo, S. S. Hur, S. E. Murthy, S. M. Cahalan, J. Xu, J. Mathur, M. Bandell, B. Coste, Y. S. Li, S. Chien, and A. Patapoutian. 2014. Piezo1, a mechanically activated ion channel, is required for vascular development in mice. Proc Natl Acad Sci USA. 111:10347-10352.
Ridone, P., E. Pandzic, M. Vassalli, C. D. Cox, A. Macmillan, P. A. Gottlieb, and B. Martinac. 2020. Disruption of membrane cholesterol organization impairs the activity of PIEZO1 channel clusters. J Gen Physiol. 152.
Ridone, P., M. Vassalli, and B. Martinac. 2019. Piezo1 mechanosensitive channels: what are they and why are they important. Biophys Rev. 11:795-805.
Rizzuto, R., D. De Stefani, A. Raffaello, and C. Mammucari. 2012. Mitochondria as sensors and regulators of calcium signalling. Nat Rev Mol Cell Biol. 13:566-578.
Romac, J. M., R. A. Shahid, S. M. Swain, S. R. Vigna, and R. A. Liddle. 2018. Piezo1 is a mechanically activated ion channel and mediates pressure induced pancreatitis. Nat Commun. 9:1715.
Romero, L. O., A. E. Massey, A. D. Mata-Daboin, F. J. Sierra-Valdez, S. C. Chauhan, J. F. Cordero-Morales. and V. Vasquez. 2019. Dietary fatty acids fine-tune Piezo1 mechanical response. Nat Commun. 10:1200.
Rosenblatt, J., M. C. Raff, and L. P. Cramer. 2001. An epithelial cell destined for apoptosis signals its neighbors to extrude it by an actin- and myosin-dependent mechanism. Curr Biol. 11:1847-1857.
Schmitz-Valckenberg, S., S. Sadda, G. Staurenghi, E. Y. Chew, M. Fleckenstein, F. G. Holz, and C. A. M. Group. 2016. GEOGRAPHIC ATROPHY: Semantic Considerations and Literature Review. Retina. 36:2250-2264.
Shi, J., A. J. Hyman, D. De Vecchis, J. Chong, L. Lichtenstein, T. S. Futers, M. Rouahi, A. N. Salvayre, N. Auge, A. C. Kalli, and D. J. Becch. 2020. Sphingomyelinase Disables Inactivation in Endogenous PIEZO1 Channels. Cell reports. 33:108225.
So, S., Y. Lee, J. Choi, S. Kang, J. Y. Lee, J. Hwang, J. Shin, J. R. Dutton, E. J. Sco, B. H. Lec, C. J. Kim, S. Mitalipov, S. J. Oh, and E. Kang. 2020. The Rho-associated kinase inhibitor fasudil can replace Y-27632 for use in human pluripotent stem cell research. PLOS One. 15:00233057.
Solis, A. G., P. Bielecki, H. R. Steach, L. Sharma, C. C. D. Harman, S. Yun, M. R. de Zoete, J. N. Warnock, S. D. F. To, A. G. York, M. Mack, M. A. Schwartz, C. S. Dela Cruz, N. W. Palm, R. Jackson, and R. A. Flavell. 2019. Mechanosensation of cyclical force by PIEZO1 is essential for innate immunity. Nature. 573:69-74.
Song, Y., D. Li, O. Farrelly, L. Miles, F. Li, S. E. Kim, T. Y. Lo, F. Wang, T. Li, K. L. Thompson-Peer, J. Gong, S. E. Murthy, B. Coste, N. Yakubovich, A. Patapoutian, Y. Xiang, P. Rompolas, L. Y. Jan. and Y. N. Jan. 2019. The Mechanosensitive Ion Channel Piezo Inhibits Axon Regeneration. Neuron. 102:373-389 e376.
Stephenson, R. E., T. Higashi, I. S. Erofeev, T. R. Arnold, M. Leda, A. B. Goryachev, and A. L. Miller. 2019. Rho Flares Repair Local Tight Junction Leaks. Dev Cell. 48:445-459 e445.
Swain, S. M., J. M. Romac, R. A. Shahid, S. J. Pandol, W. Liedtke, S. R. Vigna, and R. A. Liddle. 2020. TRPV4 channel opening mediates pressure-induced pancreatitis initiated by Piezo1 activation. J Clin Invest. 130:2527-2541.
Tan, L. X., K. A. Toops, and A. Lakkaraju. 2016. Protective responses to sublytic complement in the retinal pigment epithelium. Proc Natl Acad Sci USA. 113:8789-8794.
Terashita, T., K. Kobayashi, T. Nagano, Y. Kawa, D. Tamura, K. Nakata, M. Yamamoto, M. Tachihara, H. Kamiryo, and Y. Nishimura. 2016. Administration of JTE013 abrogates experimental asthma by regulating proinflammatory cytokine production from bronchial epithelial cells. Respir Res. 17:146.
Toops, K. A., L. X. Tan, Z. Jiang, R. Radu, and A. Lakkaraju. 2015. Cholesterol-mediated activation of acid sphingomyelinase disrupts autophagy in the retinal pigment epithelium. Mol Biol Cell. 26:1-14.
Toops, K. A., L. X. Tan, and A. Lakkaraju. 2014. A detailed three-step protocol for live imaging of intracellular traffic in polarized primary porcine RPE monolayers. Exp Eye Res. 124C:74-85.
Velasco-Estevez, M., S. O. Rolle, M. Mampay, K. K. Dev, and G. K. Sheridan. 2020. Piezo1 regulates calcium oscillations and cytokine release from astrocytes. Glia. 68:145-160.
Wang, J., Y. Ma, F. Sachs, J. Li, and T. M. Suchyna. 2016. GsMTx4-D is a cardioprotectant against myocardial infarction during ischemia and reperfusion. J Mol Cell Cardiol. 98:83-94.
Wang, J., C. Zibetti, P. Shang, S. R. Sripathi, P. Zhang, M. Cano, T. Hoang, S. Xia, H. Ji, S. L. Merbs, D. J. Zack, J. T. Handa, D. Sinha, S. Blackshaw, and J. Qian. 2018. ATAC-Seq analysis reveals a widespread decrease of chromatin accessibility in age-related macular degeneration. Nat Commun. 9:1364.
Wang, X., B. Cai, X. Yang, O. O. Sonubi, Z. Zheng, R. Ramakrishnan, H. Shi, L. Valenti, U. B. Pajvani, J. Sandhu, R. E. Infante, A. Radhakrishnan, D. F. Covey, K. L. Guan, J. Buck, L. R. Levin, P. Tontonoz, R. F. Schwabe, and I. Tabas. 2020. Cholesterol Stabilizes TAZ in Hepatocytes to Promote Experimental Non-alcoholic Steatohepatitis. Cell Metab. 31:969-986 e967.
Wong, W. L., X. Su, X. Li, C. M. Cheung, R. Klein, C. Y. Cheng, and T. Y. Wong. 2014. Global prevalence of age-related macular degeneration and disease burden projection for 2020 and 2040: a systematic review and meta-analysis. The Lancet. Global health. 2:e106-116.
Xiao, B. 2020. Levering Mechanically Activated Piezo Channels for Potential Pharmacological Intervention. Annu Rev Pharmacol Toxicol. 60:195-218.
Zanzottera, E. C., T. Ach, C. Huisingh, J. D. Messinger, K. B. Freund, and C. A. Curcio. 2016. Visualizing Retinal Pigment Epithelium Phenotypes in the Transition to Atrophy in Neovascular Age-Related Macular Degeneration. Retina. 36 Suppl 1:S26-S39.
Zhao, P. Y., G. Gan, S. Peng, S. B. Wang, B. Chen, R. A. Adelman, and L. J. Rizzolo. 2015. TRP Channels Localize to Subdomains of the Apical Plasma Membrane in Human Fetal Retinal Pigment Epithelium. Invest Ophthalmol Vis Sci. 56:1916-1923.
Zolotarevsky, Y., G. Hecht, A. Koutsouris, D. E. Gonzalez, C. Quan, J. Tom, R. J. Mrsny, and J. R. Turner. 2002. A membrane-permeant peptide that inhibits MLC kinase restores barrier function in in vitro models of intestinal disease. Gastroenterology. 123:163-172.

All publications, patents, patent applications, and accession numbers cited in this specification are herein incorporated by reference as if each individual citation were specifically and individually indicated to be incorporated by reference and are incorporated by reference with respect to the methods and/or materials in connection with which the publications are cited.

1. A method of treating or preventing macular degeneration in a subject, the method comprising administering a Piezo1 inhibitor to the subject.

2. The method of claim 1, wherein the Piezo1 inhibitor is a peptide comprising amino acid sequence SEQ ID NO:1, or a variant thereof having 1, 2, 3, 4, or 5 mutations relative to the sequence of SEQ ID NO:1.

3. The method of claim 1, wherein the peptide comprises amino acid sequence SEQ ID NO:1.

4. The method of claim 1, wherein the peptide is administered in the form of an expression vector that encodes the peptide.

5. The method of claim 1, wherein the Piezo1 inhibitor is an shRNA, siRNA, or miRNA that specifically targets Piezo1.

6. The method of claim 5, wherein the shRNA, siRNa, or miRNA is encoded by an viral vector.

7. The method of claim 1, wherein the Piezo1 inhibitor is administered as an eye drop formulation.

8. The method of claim 1, wherein the Piezo1 inhibitor is administered by local injection into the eye.

9. The method of claim 1, wherein the subject is a human.

10. The method of claim 9, wherein the subject has age-related macular degeneration (AMD), autosomal dominant or recessive Stargardt macular degeneration, Best vitelliform dystrophy, or Cone-Rod dystrophy.

11. The method of claim 10, wherein the subject has AMD or autosomal dominant or recessive Stargardt macular degeneration.

12. An eye drop formulation comprising a peptide comprising amino acid sequence SEQ ID NO:1, or a variant thereof having 1, 2, 3, 4, or 5 mutations relative to the sequence of SEQ ID NO:1.

13. The eye drop formulation of claim 12, wherein the peptide comprises amino acid sequence SEQ ID NO:1.


SEQUENCE LISTING (4 sequences)

SEQ ID NO: 1 - protein, 34 aa, Grammostola rosea
GCLEFWWKCNPNDDKCCRPKLKCSKLFKLCNFSF

SEQ ID NO: 2 - protein, 34 aa, Artificial Sequence
  Description of Artificial Sequence Modified vemon
  misc_feature (32)..(34): Xaa can be any naturally occurring amino acid
GCLEFWWKCNPNDDKCCRPKLKCSKLFKLCNXXX

SEQ ID NO: 3 - protein, 34 aa, Artificial Sequence
  Description of Artificial Sequence Modified vemon
GCLEFWWKCNPNDDKCCRPKLKCSKLFKLCNYSF

SEQ ID NO: 4 - protein, 34 aa, Artificial Sequence
  Description of Artificial Sequence Modified vemon
GCLEFWWKCNPNDDKCCRPKLKCSKLFKLCNFCF